(19) United States
(12) Patent Application Publication
(10) Pub. No.: US 2022/0375601 A1
(43) Pub. Date: Nov. 24, 2022

(54) ARTIFICIAL INTELLIGENCE BASED SYSTEMS AND METHODS FOR ANALYZING USER-SPECIFIC SKIN OR HAIR DATA TO PREDICT USER-SPECIFIC SKIN OR HAIR CONDITIONS

(71) Applicant: The Procter & Gamble Company, Cincinnati, OH (US)

(72) Inventors: Supriya Punyani, Singapore (SG); Marc Paul Lorenzi, Singapore (SG); Zelun Sun, Singapore (SG)

(21) Appl. No.: 17/326,505

(22) Filed: May 21, 2021

(51) Int. Cl. G16H 50/20; G16H 50/30; A61B 5/00; G06T 7/00

(52) U.S. Cl. CPC G16H 50/20; G16H 50/30; A61B 5/446; A61B 5/7267; G06T 7/0012; G06T 2207/30088; G06T 2207/20081; G06T 2207/20084

(57) ABSTRACT

Artificial intelligence based systems and methods for analyzing user-specific data to predict user-specific skin or hair conditions. User-specific data of a user is received at a scalp and hair analysis application and defines a scalp or hair region of the user including last wash data of the user and at least one other factor of the user. An artificial intelligence based learning model, trained with training data regarding scalp and hair regions of respective individuals, analyzes the user-specific data to generate a scalp or hair prediction value corresponding to the scalp or hair region of the user. The application generates, based on the scalp or hair prediction value, a user-specific treatment designed to address at least one feature based on the scalp or hair prediction value of the user's scalp or hair region.

### FIELD

The present disclosure generally relates to artificial intelligence (AI) based systems and methods, and more particularly to, AI based systems and methods for analyzing user-specific skin or hair data to predict user-specific skin or hair conditions.

### BACKGROUND

Generally, multiple endogenous factors of human hair and skin, such as sebum and sweat, have a real-world impact on the overall condition of a user's scalp, which may include scalp skin conditions (e.g., sebum residue, scalp skin stress) and follicle/hair conditions (e.g., hair stress, acne, scalp plugs). Additional exogenous factors, such as wind, humidity, and/or usage of various hair-related products, may also affect the condition of a user's scalp. Moreover, the user's perception of scalp related issues typically does not reflect such underlying endogenous and/or exogenous factors.

Thus a problem arises given the number of endogenous and/or exogenous factors in conjunction with the complexity of scalp and hair types, especially when considered across different users, each of whom may be associated with different demographics, races, and ethnicities. This creates a problem in the diagnosis and treatment of various human scalp conditions and characteristics. For example, prior art methods, attempting to aid a user in self-diagnosing scalp conditions generally lack sufficient information to generate accurate, user-specific diagnoses, and as a result, offer broad, overly-simplistic recommendations. Further, a user may attempt to empirically experiment with various products or techniques, but without achieving satisfactory results and/or causing possible negative side effects, impacting the condition or otherwise visual appearance of his or her scalp.

For the foregoing reasons, there is a need for AI based systems and methods for analyzing user-specific skin or hair data to predict user-specific skin or hair conditions.

### SUMMARY

Generally, as described herein, artificial intelligence (AI) based systems and methods are described for analyzing user-specific skin or hair data to predict user-specific skin or hair conditions. In some aspects, AI based systems and methods herein are configured to train AI models to input user-specific data to predict the scalp sebum of a user's scalp. Such AI based systems provide an AI based solution for overcoming problems that arise from the difficulties in identifying and treating various endogenous and/or exogenous factors or attributes affecting the condition of a human scalp, skin, and/or hair.

Generally, the AI based systems as described herein allow a user to submit user-specific data to a server(s) (e.g., including its one or more processors), or otherwise a computing device (e.g., such as locally on the user's mobile device), where the server(s) or user computing device, implements or executes an AI based learning model trained with training data of potentially thousands of instances (or more) of user-specific data regarding scalp and hair regions of respective individuals. The AI learning model may generate, based on a scalp or hair prediction value, a user-specific treatment designed to address at least one feature based on the scalp or hair prediction value of the user's scalp or hair region. For example, the user-specific data may comprise responses or other inputs indicative of scalp dryness, scalp oiliness, dandruff, stiffness, redness, unpleasant odor, itchiness, unruliness, hair fall, hair volume, thinning, detangling, hair oiliness, dryness, hair odor, acne, scalp plugs, and/or other scalp or hair factors of a specific user's scalp or hair regions. In some aspects, the user-specific treatment (and/or product specific recommendation/treatment) may be transmitted via a computer network to a user computing device of the user for rendering on a display screen. In other aspects, no transmission to the imaging server of the user-specific data occurs, where the user-specific treatment (and/or product specific recommendation/treatment) may instead be generated by the AI based learning model, executing and/or implemented locally on the user's mobile device and rendered, by a processor of the mobile device, on a display screen of the mobile device. In various aspects, such rendering may include graphical representations, overlays, annotations, and the like for addressing the feature based on the scalp or hair prediction value of the user's scalp or hair region.

In certain aspects, the AI based systems as described herein also allow a user to submit an image of the user to imaging server(s) (e.g., including its one or more processors), or otherwise a computing device (e.g., such as locally on the user's mobile device), where the imaging server(s) or user computing device, implements or executes an AI based learning model trained with pixel data of potentially 10,000s (or more) images depicting scalp or hair regions of respective individuals. The AI based learning model may generate, based on a scalp or hair prediction value, a user-specific treatment designed to address at least one feature identifiable within the pixel data comprising the user's scalp or hair region. For example, a portion of a user's scalp or hair region can comprise pixels or pixel data indicative of white sebum, scalp dryness, scalp oiliness, dandruff, stiffness, redness/irritation, itchiness, unruliness, hair fall, hair volume, thinning, detangling, hair oiliness, dryness, hair odor, acne, scalp plugs, and/or other scalp or hair factors of a specific user's scalp or hair regions. In some aspects, the user-specific treatment (and/or product specific recommendation/treatment) may be transmitted via a computer network to a user computing device of the user for rendering on a display screen. In other aspects, no transmission to the imaging server of the image of the user occurs, where the user-specific treatment (and/or product specific recommendation/treatment) may instead be generated by the AI based learning model, executing and/or implemented locally on the user's mobile device and rendered, by a processor of the mobile device, on a display screen of the mobile device. In various aspects, such rendering may include graphical representations, overlays, annotations, and the like for addressing the feature in the pixel data.

More specifically, as described herein, an AI based system is disclosed. The AI based learning system is configured to analyze user-specific skin or hair data (also referenced herein as “user-specific data”) to predict user-specific skin or hair conditions (also referenced herein as “scalp or hair prediction values” and “scalp and hair condition values”). The AI based system comprises one or more processors, a scalp and hair analysis application (app) comprising computing instructions configured to execute on the one or more processors, and an AI based learning model. The AI based learning model is accessible by the scalp and hair analysis app, and is trained with training data regarding scalp and hair regions of respective individuals. The AI based learning model is configured to output one or more scalp or hair predictions corresponding to one or more features of the scalp or hair regions of respective individuals. The training data regarding scalp and hair regions of respective individuals is selected from one or more values corresponding to last wash data of the respective individuals and at least one of: one or more of scalp factors, one or more hair factors, or wash frequency. The training data comprises data generated with a scalp or hair measurement device configured to determine the one or more features of the scalp or hair regions. The computing instructions of the scalp and hair analysis app when executed by the one or more processors, cause the one or more processors to: receive user-specific data of a user, the user-specific data defining a scalp or hair region of the user comprising (1) last wash data of the user, and (2) at least one of: one or more scalp factors of the user, one or more hair factors of the user, or a wash frequency of the user, analyze, by the AI based learning model, the user-specific data to generate a scalp or hair prediction value corresponding to the scalp or hair region of the user, and generate, based on the scalp or hair prediction value, a user-specific treatment designed to address at least one feature based on the scalp or hair prediction value of the user's scalp or hair region.

In addition, as described herein, an artificial intelligence (AI) based method is disclosed for analyzing user-specific skin or hair data to predict user-specific skin or hair conditions. The AI based method comprises receiving, at a scalp and hair analysis application (app) executing on one or more processors, user-specific data of a user, the user-specific data defining a scalp or hair region of the user comprising (1) last wash data of the user, and (2) at least one of: one or more scalp factors of the user, one or more hair factors of the user, or a wash frequency of the user; analyzing, by an artificial intelligence (AI) based learning model accessible by the scalp and hair analysis app, the user-specific data to generate a scalp or hair prediction value corresponding to the scalp or hair region of the user, wherein the AI based learning model is trained with training data regarding scalp and hair regions of respective individuals and is configured to output one or more scalp or hair predictions corresponding to one or more features of the scalp or hair regions of respective individuals, wherein the training data regarding scalp and hair regions of respective individuals is selected from one or more values corresponding to last wash data of the respective individuals and at least one of: one or more of scalp factors, one or more hair factors, or wash frequency, wherein the training data comprises data generated with a scalp or hair measurement device configured to determine the one or more features of the scalp or hair regions; and generating, by the scalp and hair analysis app based on the scalp or hair prediction value, a user-specific treatment designed to address at least one feature based on the scalp or hair prediction value of the user's scalp or hair region.

Further, as described herein, a tangible, non-transitory computer-readable medium storing instructions for analyzing user-specific skin or hair data to predict user-specific skin or hair conditions is disclosed. The instructions, when executed by one or more processors, may cause the one or more processors to: receive, at a scalp and hair analysis application (app) executing on one or more processors, user-specific data of a user, the user-specific data defining a scalp or hair region of the user comprising (1) last wash data of the user, and (2) at least one of: one or more scalp factors of the user, one or more hair factors of the user, or a wash frequency of the user; analyze, by an artificial intelligence (AI) based learning model accessible by the scalp and hair analysis app, the user-specific data to generate a scalp or hair prediction value corresponding to the scalp or hair region of the user, wherein the AI based learning model is trained with training data regarding scalp and hair regions of respective individuals and is configured to output one or more scalp or hair predictions corresponding to one or more features of the scalp or hair regions of respective individuals, wherein the training data regarding scalp and hair regions of respective individuals is selected from one or more values corresponding to last wash data of the respective individuals and at least one of: one or more of scalp factors, one or more hair factors, or wash frequency, wherein the training data comprises data generated with a scalp or hair measurement device configured to determine the one or more features of the scalp or hair regions; and generate, by the scalp and hair analysis app based on the scalp or hair prediction value, a user-specific treatment designed to address at least one feature based on the scalp or hair prediction value of the user's scalp or hair region.

In accordance with the above, and with the disclosure herein, the present disclosure includes improvements in computer functionality or in improvements to other technologies at least because the disclosure describes that, e.g., a server, or otherwise computing device (e.g., a user computer device), is improved where the intelligence or predictive ability of the server or computing device is enhanced by a trained (e.g., machine learning trained) AI based learning model. The AI based learning model, executing on the server or computing device, is able to more accurately identify, based on user-specific data of other individuals, one or more of a user-specific scalp or hair region feature, a scalp or hair prediction value, and/or a user-specific treatment designed to address at least one feature based on the scalp or hair prediction value of the user's scalp or hair region. That is, the present disclosure describes improvements in the functioning of the computer itself or “any other technology or technical field” because a server or user computing device is enhanced with a plurality of training data (e.g., potentially thousands of instances (or more) of user-specific data regarding scalp and hair regions of respective individuals) to accurately predict, detect, or determine user skin or hair conditions based on user-specific data, such as newly provided customer responses/inputs/images. This improves over the prior art at least because existing systems lack such predictive or classification functionality and are simply not capable of accurately analyzing user-specific data to output a predictive result to address at least one feature based on the scalp or hair prediction value of the user's scalp or hair region.

Specifically, the systems and methods of the present disclosure feature improvements over conventional techniques by training the AI based learning model with a plurality of clinical data related to scalp and hair conditions (e.g., scalp sebum and scalp stress data) of a plurality of individuals. The clinical data generally includes an individual's self-assessment of the individual's scalp and hair condition in the form of textual questionnaire responses for each of the plurality of individuals, and physical measurements corresponding to each individual's scalp and hair (e.g., collected with a scalp or hair measurement device). Once trained using the clinical data, the AI based learning model provides high-accuracy scalp and hair condition predictions for a user, without requiring an image of the user, to a degree that is unattainable using conventional techniques. In fact, the AI based systems of the present disclosure achieve approximately 75% accuracy when predicting scalp and hair condition values for users based upon user-specific data (e.g., responses to a questionnaire), reflecting a substantial correlation between the AI based learning model and the user's actual scalp and hair condition that conventional techniques simply cannot not achieve. Moreover, in certain aspects, the clinical data includes user-specific images corresponding to the self-assessment of each individual of the plurality of individuals, and the user additionally submits a user-specific image as part of the user-specific data. In these aspects, the accuracy of the AI based learning model is further increased, providing incredibly high-accuracy scalp and hair predictions for users that conventional techniques are incapable of providing.

For similar reasons, the present disclosure relates to improvements to other technologies or technical fields at least because the present disclosure describes or introduces improvements to computing devices in the scalp and hair care field and scalp and hair care products field, whereby the trained AI based learning model executing on the imaging device(s) or computing devices improves the field of scalp and hair region care, and chemical formulations of scalp and hair care products thereof, with AI and/or digital based analysis of user-specific data and/or images to output a predictive result to address at least one feature identifiable within the user-specific data defining a scalp or hair region of the user comprising (1) last wash data of the user, and (2) at least one of: one or more scalp factors of the user, one or more hair factors of the user, or a wash frequency of the user.

Further, the present disclosure relates to improvement to other technologies or technical fields at least because the present disclosure describes or introduces improvements to computing devices in the scalp and hair care field and scalp and hair products field, whereby the trained AI based learning model executing on the computing devices and/or imaging device(s) improve the underlying computer device (e.g., server(s) and/or user computing device), where such computer devices are made more efficient by the configuration, adjustment, or adaptation of a given machine-learning network architecture. For example, in some aspects, fewer machine resources (e.g., processing cycles or memory storage) are used by decreasing computational resources by decreasing machine-learning network architecture needed to analyze images, including by reducing depth, width, image size, or other machine-learning based dimensionality requirements. Such reduction frees up the computational resources of an underlying computing system, thereby making it more efficient.

In addition, the present disclosure includes applying certain of the claim elements with, or by use of, a particular machine, e.g., a scalp or hair measurement device, which generates training data used to train the AI based learning model.

In addition, the present disclosure includes specific features other than what is well-understood, routine, conventional activity in the field, or adding unconventional steps that confine the claim to a particular useful application, e.g., analyzing user-specific data defining a scalp or hair region of a user to generate a scalp or hair prediction value and a user-specific treatment designed to address at least one feature based on the scalp or hair prediction value of the user's scalp or hair region.

Advantages will become more apparent to those of ordinary skill in the art from the following description of the preferred aspects which have been shown and described by way of illustration. As will be realized, the present aspects may be capable of other and different aspects, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts an aspect of a particular aspect of the disclosed system and methods, and that each of the Figures is intended to accord with a possible aspect thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present aspects are not limited to the precise arrangements and instrumentalities shown, wherein:

FIG. 1 illustrates an example artificial intelligence (AI) based system configured to analyze user-specific skin or hair data to predict user-specific skin or hair conditions, in accordance with various aspects disclosed herein.

FIG. 2 illustrates an example questionnaire correlation diagram that may be used for training and/or implementing an AI based learning model, in accordance with various aspects disclosed herein.

FIG. 3 illustrates an example correlation table having scalp and hair factors correlating to outputs of the AI based learning model, in accordance with various aspects disclosed herein.

FIG. 4 illustrates an AI based method for analyzing user-specific skin or hair data to predict user-specific skin or hair conditions, in accordance with various aspects disclosed herein.

FIG. 5A illustrates an example user interface as rendered on a display screen of a user computing device in accordance with various aspects disclosed herein.

FIG. 5B illustrates another example user interface as rendered on a display screen of a user computing device in accordance with various aspects disclosed herein.

The Figures depict preferred aspects for purposes of illustration only. Alternative aspects of the systems and methods illustrated herein may be employed without departing from the principles of the invention described herein.

### DETAILED DESCRIPTION OF THE INVENTION

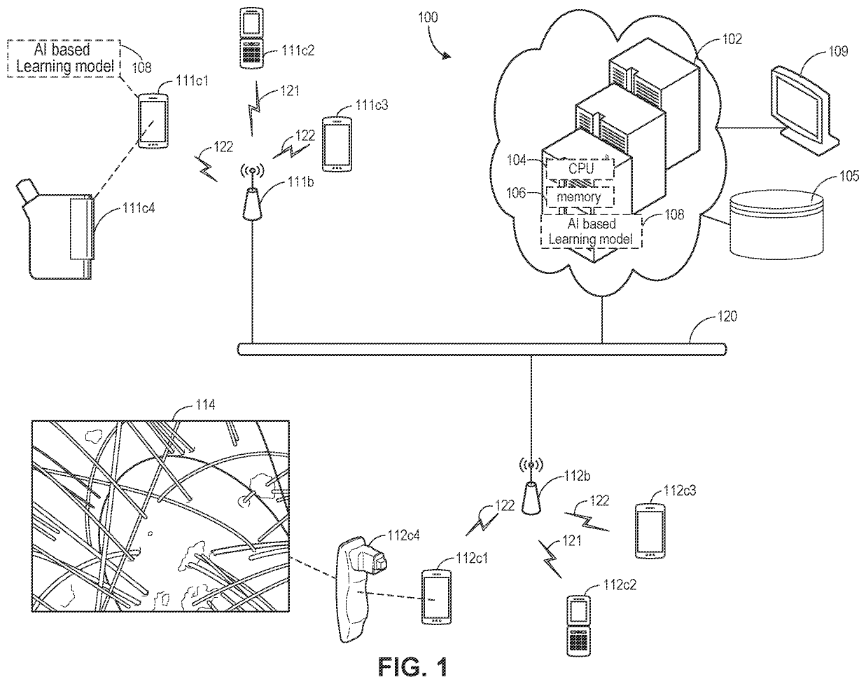

FIG. 1 illustrates an example AI based system 100 configured to analyze user-specific skin or hair data to predict user-specific skin or hair conditions, in accordance with various aspects disclosed herein. Generally, as referred to herein, the user-specific skin or hair data may include user responses/inputs related to questions/prompts presented to the user via a display and/or user interface of a user computing device that are directed to the condition of the user's scalp and/or hair. For example, the user-specific skin or hair data may include a user response indicating when the user last washed their hair (e.g., 3 hours ago, 1 day ago, 5 days ago, etc.) and that the user experiences a significant amount of scalp dryness. In certain aspects, the user-specific skin or hair data may also include images of a user's head, and more particularly, the user's scalp.

In the example aspect of FIG. 1, the AI based system 100 includes server(s) 102, which may comprise one or more computer servers. In various aspects server(s) 102 comprise multiple servers, which may comprise multiple, redundant, or replicated servers as part of a server farm. In still further aspects, server(s) 102 may be implemented as cloud-based servers, such as a cloud-based computing platform. For example, server(s) 102 may be any one or more cloud-based platform(s) such as MICROSOFT AZURE, AMAZON AWS, or the like. Server(s) 102 may include one or more processor(s) 104, one or more computer memories 106, and an AI based learning model 108.

The memories 106 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memorie(s) 106 may store an operating system (OS) (e.g., Microsoft Windows, Linux, UNIX, etc.) capable of facilitating the functionalities, apps, methods, or other software as discussed herein. The memorie(s) 106 may also store the AI based learning model 108, which may be a machine learning model, trained on various training data (e.g., potentially thousands of instances (or more) of user-specific data regarding scalp and hair regions of respective individuals) and, in certain aspects, images (e.g., image 114), as described herein. Additionally, or alternatively, the AI based learning model 108 may also be stored in database 105, which is accessible or otherwise communicatively coupled to server(s) 102. In addition, memories 106 may also store machine readable instructions, including any of one or more application(s) (e.g., a scalp and hair application as described herein), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For example, at least some of the applications, software components, or APIs may be, include, otherwise be part of, an AI based machine learning model or component, such as the AI based learning model 108, where each may be configured to facilitate their various functionalities discussed herein. It should be appreciated that one or more other applications may be envisioned and that are executed by the processor(s) 104.

The processor(s) 104 may be connected to the memories 106 via a computer bus responsible for transmitting electronic data, data packets, or otherwise electronic signals to and from the processor(s) 104 and memories 106 in order to implement or perform the machine readable instructions, methods, processes, elements or limitations, as illustrated, depicted, or described for the various flowcharts, illustrations, diagrams, figures, and/or other disclosure herein.

Processor(s) 104 may interface with memory 106 via the computer bus to execute an operating system (OS). Processor(s) 104 may also interface with the memory 106 via the computer bus to create, read, update, delete, or otherwise access or interact with the data stored in memories 106 and/or the database 104 (e.g., a relational database, such as Oracle, DB2, MySQL, or a NoSQL based database, such as MongoDB). The data stored in memories 106 and/or database 105 may include all or part of any of the data or information described herein, including, for example, training data (e.g., as collected by user computing devices 111c1-111c3 and/or 112c1-112c3 and/or the scalp or hair measurement device 111c4); images and/or user images (e.g., including image 114); and/or other information and/or images of the user, including demographic, age, race, skin type, hair type, hair style, or the like, or as otherwise described herein.

The server(s) 102 may further include a communication component configured to communicate (e.g., send and receive) data via one or more external/network port(s) to one or more networks or local terminals, such as computer network 120 and/or terminal 109 (for rendering or visualizing) described herein. In some aspects, the server(s) 102 may include a client-server platform technology such as ASP.NET, Java J2EE, Ruby on Rails, Node.js, a web service or online API, responsive for receiving and responding to electronic requests. The server(s) 102 may implement the client-server platform technology that may interact, via the computer bus, with the memories(s) 106 (including the applications(s), component(s), API(s), data, etc. stored therein) and/or database 105 to implement or perform the machine readable instructions, methods, processes, elements or limitations, as illustrated, depicted, or described for the various flowcharts, illustrations, diagrams, figures, and/or other disclosure herein.

In various aspects, the server(s) 102 may include, or interact with, one or more transceivers (e.g., WWAN, WLAN, and/or WPAN transceivers) functioning in accordance with IEEE standards, 3GPP standards, or other standards, and that may be used in receipt and transmission of data via external/network ports connected to computer network 120. In some aspects, computer network 120 may comprise a private network or local area network (LAN). Additionally, or alternatively, computer network 120 may comprise a public network such as the Internet.

The server(s) 102 may further include or implement an operator interface configured to present information to an administrator or operator and/or receive inputs from the administrator or operator. As shown in FIG. 1, an operator interface may provide a display screen (e.g., via terminal 109). The server(s) 102 may also provide I/O components (e.g., ports, capacitive or resistive touch sensitive input panels, keys, buttons, lights, LEDs), which may be directly accessible via, or attached to, imaging server(s) 102 or may be indirectly accessible via or attached to terminal 109. According to some aspects, an administrator or operator may access the server 102 via terminal 109 to review information, make changes, input training data or images, initiate training of the AI based learning model 108, and/or perform other functions.

As described herein, in some aspects, the server(s) 102 may perform the functionalities as discussed herein as part of a “cloud” network or may otherwise communicate with other hardware or software components within the cloud to send, retrieve, or otherwise analyze data or information described herein.

In general, a computer program or computer based product, application, or code (e.g., the model(s), such as AI models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 104 (e.g., working in connection with the respective operating system in memories 106) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

As shown in FIG. 1, the server(s) 102 are communicatively connected, via computer network 120 to the one or more user computing devices 111c1-111c4 and/or 112c1-112c4 via base stations 111b and 112b. In some aspects, base stations 111b and 112b may comprise cellular base stations, such as cell towers, communicating to the one or more user computing devices 111c1-111c4 and 112c1-112c4 via wireless communications 121 based on any one or more of various mobile phone standards, including NMT, GSM, CDMA, UMMTS, LTE, 5G, or the like. Additionally, or alternatively, base stations 111b and 112b may comprise routers, wireless switches, or other such wireless connection points communicating to the one or more user computing devices 111c1-111c4 and 112c1-112c4 via wireless communications 122 based on any one or more of various wireless standards, including by non-limiting example, IEEE 802.11a/b/c/g (WIFI), the BLUETOOTH standard, or the like.

Any of the one or more user computing devices 111c1-111c4 and/or 112c1-112c4 may comprise mobile devices and/or client devices for accessing and/or communications with the server(s) 102. Such client devices may comprise one or more mobile processor(s) and/or an imaging device for capturing images, such as images as described herein (e.g., image 114). In various aspects, user computing devices 111c1-111c3 and/or 112c1-112c3 may comprise a mobile phone (e.g., a cellular phone), a tablet device, a personal data assistance (PDA), or the like, including, by non-limiting example, an APPLE iPhone or iPad device or a GOOGLE ANDROID based mobile phone or table.

In certain aspects, any of the user computing devices 111c1-111c3, 112c1-112c3 may include an integrated camera configured to capture image data comprising one or more sebum images defining an amount of human sebum identifiable within pixel data of the one or more sebum images. For example, the user computing device 111c3 may be a smartphone with an integrated camera including a lens that a user may apply (referenced herein as “tapping”) to the user's skin surface (e.g., scalp or hair) to distribute sebum on the camera lens, and thereby capture image data containing the one or more sebum images. In these examples, the user computing device 111c3 may include instructions that cause the processor of the device 111c3 to analyze the captured sebum images and determine an amount and/or a type of human sebum represented in the captured sebum images. Further, in these examples, the sebum images may not include fully resolved visualizations, but instead may feature sebum patterns that the processor of the device 111c3 may analyze and match with known sebum patterns/distributions to determine an amount of sebum distributed over the camera lens. The processor of the device 111c3 may thereby extrapolate the amount of sebum distributed over the camera lens to determine a likely amount of sebum distributed over the user's scalp/forehead.

In certain aspects, the user computing device 111c4 may be a scalp or hair measurement device that a user may use to measure one or more factors of the user's scalp or hair. Specifically, the scalp or hair measurement device 111c4 may include a probe or other apparatus configured to apply a reactive tape or other substrate to a user's skin surface. The reactive tape or other substrate may absorb or otherwise lift oil (e.g., sebum) from the user's skin surface that can then be quantitatively measured using an optical measurement process based on the amount and types of residue present on the reactive tape or other substrate. As a particular example, the scalp or hair measurement device 111c4 may be the SEBUMETER SM 815 device, developed by COURAGE+KHAZAKA ELECTRONIC GMBH. In this example, the user may apply the probe with the mat tape to the user's scalp or hair to apply sebum to the mat tape. The user may then evaluate the sebum content present on the tape using grease spot photometry to determine sebum levels of the user's scalp or hair.

In additional aspects, the user computing device 112c4 may be a portable microscope device that a user may use to capture detailed images of the user's scalp or hair. Specifically, the portable microscope device 112c4 may include a microscopic camera that is configured to capture images (e.g., any one or more of images 202a, 202b, and/or 202c) at an approximately microscopic level of a user's scalp or hair regions. For example, unlike any of the user computing devices 111c1-111c4 and 112c1-112c3, the portable microscope device 112c4 may capture detailed, high-magnification (e.g., 2 megapixels for 60-200 times magnification) images of the user's scalp or hair regions while maintaining physical contact with the user's scalp or hair. As a particular example, the portable microscope device 112c4 may be the API 202 HAIR SCALP ANALYSIS device, developed by ARAM HUVIS. In certain aspects, the portable microscope device 112c4 may also include a display or user interface configured to display the captured images and/or the results of the image analysis to the user.

Additionally, or alternatively, the scalp or hair measurement device 111c4 and/or the portable microscope device 112c4 may be communicatively coupled to a user computing device 111c1, 112c1 (e.g., a user's mobile phone) via a WiFi connection, a BLUETOOTH connection, and/or any other suitable wireless connection, and the scalp or hair measurement device 111c4 and/or the portable microscope device 112c4 may be compatible with a variety of operating platforms (e.g., Windows, iOS, Android, etc.). Thus, the scalp or hair measurement device 111c4 and/or the portable microscope device 112c4 may transmit the user's scalp or hair factors and/or the captured images to the user computing devices 111c1, 112c1 for analysis and/or display to the user. Moreover, the portable microscope device 112c4 may be configured to capture high-quality video of a user's scalp, and may stream the high-quality video of the user's scalp to a display of the portable microscope device 112c4 and/or a communicatively coupled user computing device 112c1 (e.g., a user's mobile phone). In certain additional aspects, the components of each of the scalp or hair measurement device 111c4 and/or the portable microscope device 112c4 and the communicatively connected user computing device 111c1, 112c1 may be incorporated into a singular device.

In additional aspects, user computing devices 111c1-111c3 and/or 112c1-112c3 may comprise a retail computing device. A retail computing device may comprise a user computer device configured in a same or similar manner as a mobile device, e.g., as described herein for user computing devices 111c1-111c3 and 112c1-112c3, including having a processor and memory, for implementing, or communicating with (e.g., via server(s) 102), an AI based learning model 108 as described herein. Additionally, or alternatively, a retail computing device may be located, installed, or otherwise positioned within a retail environment to allow users and/or customers of the retail environment to utilize the AI based systems and methods on site within the retail environment. For example, the retail computing device may be installed within a kiosk for access by a user. The user may then provide responses to a questionnaire and/or upload or transfer images (e.g., from a user mobile device) to the kiosk to implement the AI based systems and methods described herein. Additionally, or alternatively, the kiosk may be configured with a camera to allow the user to take new images (e.g., in a private manner where warranted) of himself or herself for upload and transfer. In such aspects, the user or consumer himself or herself would be able to use the retail computing device to receive and/or have rendered a user-specific treatment related to the user's scalp or hair region, as described herein, on a display screen of the retail computing device.

Additionally, or alternatively, the retail computing device may be a mobile device (as described herein) as carried by an employee or other personnel of the retail environment for interacting with users or consumers on site. In such aspects, a user or consumer may be able to interact with an employee or otherwise personnel of the retail environment, via the retail computing device (e.g., by providing responses to a questionnaire, transferring images from a mobile device of the user to the retail computing device, or by capturing new images by a camera of the retail computing device), to receive and/or have rendered a user-specific treatment related to the user's scalp or hair region, as described herein, on a display screen of the retail computing device.

In various aspects, the one or more user computing devices 111c1-111c3 and/or 112c1-112c4 may implement or execute an operating system (OS) or mobile platform such as APPLE's iOS and/or GOOGLE's ANDROID operation system. Any of the one or more user computing devices 111c1-111c3 and/or 112c1-112c3 may comprise one or more processors and/or one or more memories for storing, implementing, or executing computing instructions or code, e.g., a mobile application or a home or personal assistant application, as described in various aspects herein. As shown in FIG. 1, the AI based learning model 108 and/or an imaging application as described herein, or at least portions thereof, may also be stored locally on a memory of a user computing device (e.g., user computing device 111c1).

User computing devices 111c1-111c4 and/or 112c1-112c4 may comprise a wireless transceiver to receive and transmit wireless communications 121 and/or 122 to and from base stations 111b and/or 112b. In various aspects, user-specific data (e.g., user responses/inputs to questionnaire(s) presented on user computing device 111c1, measurement data acquired by the scalp or hair measurement device 111c4) and/or pixel based images (e.g., image 114) may be transmitted via computer network 120 to the server(s) 102 for training of model(s) (e.g., AI based learning model 108) and/or analysis as described herein.

In addition, the one or more user computing devices 111c1-111c3 and/or 112c1-112c4 may include an imaging device and/or digital video camera for capturing or taking digital images and/or frames (e.g., image 114). Each digital image may comprise pixel data for training or implementing model(s), such as AI or machine learning models, as described herein. For example, an imaging device and/or digital video camera of, e.g., any of user computing devices 111c1-111c3 and/or 112c1-112c4, may be configured to take, capture, or otherwise generate digital images (e.g., pixel based image 114) and, at least in some aspects, may store such images in a memory of a respective user computing devices. Additionally, or alternatively, such digital images may also be transmitted to and/or stored on memorie(s) 106 and/or database 105 of server(s) 102.

Still further, each of the one or more user computer devices 111c1-111c3 and/or 112c1-112c4 may include a display screen for displaying graphics, images, text, products, user-specific treatments, data, pixels, features, and/or other such visualizations or information as described herein. In various aspects, graphics, images, text, products, user-specific treatments, data, pixels, features, and/or other such visualizations or information may be received from the server(s) 102 for display on the display screen of any one or more of user computer devices 111c1-111c3 and/or 112c1-112c4. Additionally, or alternatively, a user computing device may comprise, implement, have access to, render, or otherwise expose, at least in part, an interface or a guided user interface (GUI) for displaying text and/or images on its display screen.

In some aspects, computing instructions and/or applications executing at the server (e.g., server(s) 102) and/or at a mobile device (e.g., mobile device 111c1) may be communicatively connected for analyzing user-specific data defining a scalp or hair region of the user comprising (1) last wash data of the user, and (2) at least one of: one or more scalp factors of the user, one or more hair factors of the user, or a wash frequency of the user to generate a user-specific treatment, as described herein. For example, one or more processors (e.g., processor(s) 104) of server(s) 102 may be communicatively coupled to a mobile device via a computer network (e.g., computer network 120). For ease of discussion, the user-specific data and the last wash data may be collectively referenced herein as “user-specific data”.

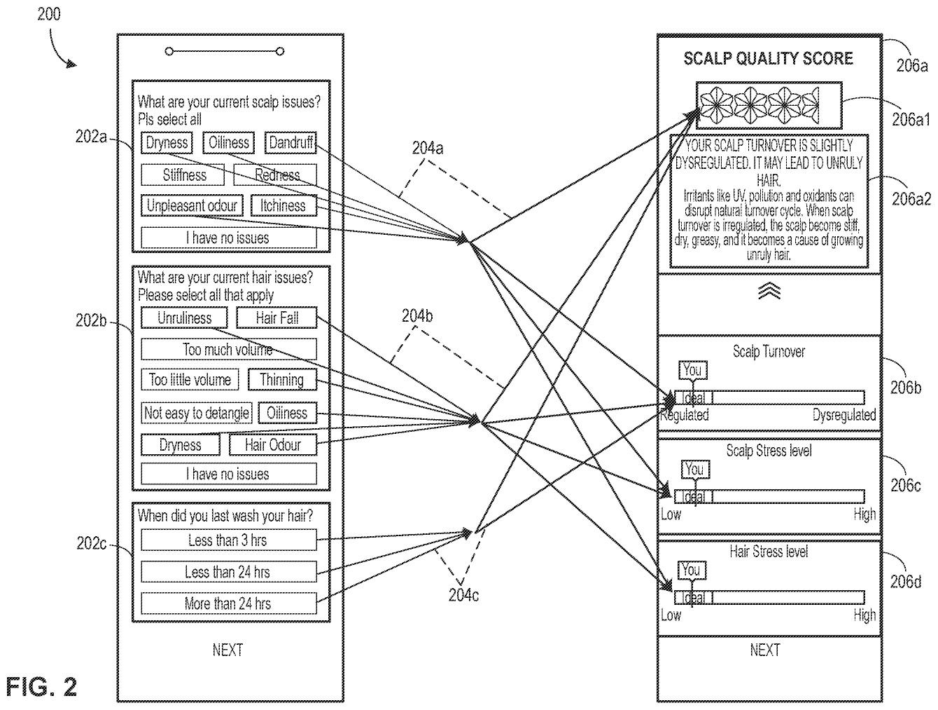

FIG. 2 illustrates an example questionnaire correlation diagram 200 that may be used for training and/or implementing an AI based learning model, in accordance with various aspects disclosed herein. Generally, the questionnaire correlation diagram 200 may represent or correspond to user data or information as input or used by the various correlations determined and/or utilized by the AI based learning model to associate the user-specific data (e.g., each of the scalp factor section 202a, the hair factor section 202b, and the last wash section 202c) and the scalp and hair prediction values (e.g., each of the scalp quality score section 206a, the scalp turnover section 206b, the scalp stress level section 206c, and the hair stress level section 206d). A user may execute a scalp and hair analysis application (app), which in turn, may display a user interface that may include sections/prompts similar to those provided in sections 202a, 202b, and/or 202c. When the user indicates one or more of the factors included in one or more of the sections, the AI based learning model may analyze the user-specific data to generate the scalp and hair prediction values, and the scalp and hair analysis app may render a user interface that may include sections similar to the sections 206a, 206b, 206c, and/or 206d.

The user-specific data may include a scalp factor section 202a that prompts the user to provide, and provides options for the user to indicate, user-specific data directed to one or more scalp factors of the user's scalp. For example, the scalp factor section 202a may query the user whether or not the user experiences any scalp issues, and may request that the user select one or more of the options presented as part of the scalp factor section 202a. The one or more options (e.g., scalp factors) may include, for example, scalp dryness, scalp oiliness, dandruff, stiffness, redness, unpleasant scalp odor, itchiness, no perceived issues, and/or any other suitable scalp factors or combinations thereof. A user may indicate each applicable scalp factor through, for example, interaction with a user interface of a scalp and hair analysis app executing on the user's computing device (e.g., user computing device 111c1). For each scalp factor indicated by the user, the AI based learning model may incorporate the indicated scalp factor as part of the analysis of the user-specific data to generate the user's scalp and hair prediction values (e.g., each of 206a, 206b, 206c, 206d). Namely, the scalp factor correlations 204a illustrate the multiple correlations the AI based learning model may determine and/or utilize to generate the user's scalp and hair prediction values based, in part, upon the indicated scalp factors.

Additionally, the user-specific data may include a hair factor section 202b that prompts the user to provide, and provides options for the user to indicate, user-specific data directed to one or more hair factors of the user's hair. For example, the hair factor section 202b may query the user whether or not the user experiences any hair issues, and may request that the user select one or more of the options presented as part of the hair factor section 202b. The one or more options (e.g., hair factors) may include, for example, unruliness, hair fall, hair volume, thinning, detangling, hair oiliness, dryness, hair odor, no perceived issues, and/or any other suitable hair factors or combinations thereof. A user may indicate each applicable hair factor through, for example, interaction with a user interface of a scalp and hair analysis app executing on the user's computing device (e.g., user computing device 111c1). For each hair factor indicated by the user, the AI based learning model may incorporate the indicated hair factor as part of the analysis of the user-specific data to generate the user's scalp and hair prediction values (e.g., each of 206a, 206b, 206c, 206d). Namely, the hair factor correlations 204b illustrate the multiple correlations the AI based learning model may determine and/or utilize to generate the user's scalp and hair prediction values based, in part, upon the indicated hair factors.

Further, the user-specific data may include a last wash section 202c that prompts the user to provide, and provides options for the user to indicate, user-specific data directed to a last wash of the user's hair. Generally, scalp and hair sebum as well as other features build-up and/or change substantially over time based on when the user last washed their hair. Thus, each of the scalp or hair predictions output by the AI based learning model are influenced significantly by the user's response to the last wash section 202c. For example, the last wash section 202c may query the user regarding the last time the user washed their hair, and may request that the user select one or more of the options presented as part of the last wash section 202c. The one or more options (e.g., a last wash) may include, for example, less than 3 hours prior to providing responses/inputs to the questionnaire, less than 24 hours prior to providing responses/inputs to the questionnaire, more than 24 hours prior to providing responses/inputs to the questionnaire, and/or any other suitable last wash data or combinations thereof. Of course, it is to be understood that the one or more options presented in the last wash section 202c may include any suitable option for a user to input when the user last washed their hair, such as a sliding scale and/or a manually-entered (e.g., by typing on a keyboard or virtually rendered keyboard on the user's mobile device) numerical value indicating the number of hours, days, etc. since the user last washed their hair.

In any event, a user may indicate an applicable last wash option through, for example, interaction with a user interface of a scalp and hair analysis app executing on the user's computing device (e.g., user computing device 111c1). When the user indicates a last wash option, the AI based learning model may incorporate the indicated last wash option as part of the analysis of the user-specific data to generate the user's scalp and hair prediction values (e.g., each of 206a, 206b, 206c, 206d). Namely, the last wash correlations 204c illustrate the multiple correlations the AI based learning model may determine and/or utilize to generate the user's scalp and hair prediction values based, in part, upon the indicated last wash of the user's hair.

The scalp and hair prediction values may include a scalp quality score section 206a, which may display a scalp quality score of a user. For example, as a result of the AI based learning model analyzing the user-specific data (e.g., derived from each of the scalp factor section 202a, the hair factor section 202b, and/or the last wash section 202c), the AI based learning model may generate a scalp quality score of a user, as represented by the graphical score 206a1. The graphical score 206a1 may indicate to a user that the user's scalp quality score is, for example, a 3.5 out of a potential maximum score of 4. However, it is to be understood that the scalp quality score may be represented to a user as a graphical rendering (e.g., graphical score 206a1), an alphanumerical value, a color value, and/or any other suitable representation or combinations thereof.

Moreover, the AI based learning model may also generate, as part of the scalp or hair prediction values, a scalp quality score description 206a2 that may inform a user about their received scalp quality score (e.g., represented by the graphical score 206a1). The scalp and hair analysis app may render the scalp quality score description 206a2 as part of the user interface when the AI based learning model completes the analysis of the user-specific data. The scalp quality score description 206a2 may include a description of, for example, a predominant scalp/hair factor and/or last wash data leading to a reduced score, endogenous/exogenous factors causing scalp and/or hair issues, and/or any other information or combinations thereof. As an example, the scalp quality score description 206a2 may inform a user that their scalp turnover is slightly dysregulated, and as a result, the user may experience unruly hair. Further in this example, the scalp quality score description 206a2 may convey to the user that irritants such as ultraviolet (UV) radiation, pollution, and oxidants may disrupt and/or otherwise result in an unregulated natural scalp turnover cycle. Accordingly, the scalp quality score description 206a2 may indicate to a user that when scalp turnover is unregulated/dysregulated, the scalp may become stiff, dry, greasy, and may cause the user's hair to grow in an unruly fashion.

Additionally, the scalp and hair prediction values may include a scalp turnover section 206b, which may display a scalp turnover level of a user. For example, as a result of the AI based learning model analyzing the user-specific data (e.g., derived from each of the scalp factor section 202a, the hair factor section 202b, and/or the last wash section 202c), the AI based learning model may generate a scalp turnover level of a user, as represented by the sliding scale and corresponding indicator within the scalp turnover section 206b. Generally, the indicator located on the sliding scale of the scalp turnover section 206b may graphically illustrate the user's scalp turnover level to the user as between completely regulated and completely dysregulated. In the example aspect illustrated in FIG. 2, the user's scalp turnover level represented in the scalp turnover section 206b indicates that the user's scalp turnover is slightly dysregulated. The indicator may also provide a numerical representation of the user's scalp turnover, and may indicate to a user that the user's scalp turnover level is, for example, a 4.3 out of a potential maximum score of 5. Of course, the scoring scale may include any suitable minimum and/or maximum values (e.g., 0 to 5, 1 to 6, 0 to 100, etc.). Regardless, it is to be understood that the scalp turnover level may be represented to a user as a graphical rendering (e.g., the sliding scale and indicator of the scalp turnover section 206b), an alphanumerical value, a color value, and/or any other suitable representation or combinations thereof.

Further, the scalp and hair prediction values may include a scalp stress level section 206c, which may display a scalp stress level of a user. For example, as a result of the AI based learning model analyzing the user-specific data (e.g., derived from each of the scalp factor section 202a, the hair factor section 202b, and/or the last wash section 202c), the AI based learning model may generate a scalp stress level of a user, as represented by the sliding scale and corresponding indicator within the scalp stress level section 206c. Generally, the indicator located on the sliding scale of the scalp stress level section 206c may graphically illustrate the user's scalp stress level to the user as between low scalp stress and high scalp stress. In the example aspect illustrated in FIG. 2, the user's scalp stress level represented in the scalp stress level section 206c indicates that the user's scalp stress level is relatively low (e.g., within the “ideal” portion of the sliding scale). The indicator may also provide a numerical representation of the user's scalp stress level, and may indicate to a user that the user's scalp stress level is, for example, a 9.2 out of a potential maximum score of 10. Of course, the scoring scale may include any suitable minimum and/or maximum values (e.g., 0 to 5, 1 to 6, 0 to 100, etc.). Regardless, it is to be understood that the scalp stress level may be represented to a user as a graphical rendering (e.g., the sliding scale and indicator of the scalp stress level section 206c), an alphanumerical value, a color value, and/or any other suitable representation or combinations thereof.

Moreover, the scalp and hair prediction values may include a hair stress level section 206d, which may display a hair stress level of a user. For example, as a result of the AI based learning model analyzing the user-specific data (e.g., derived from each of the scalp factor section 202a, the hair factor section 202b, and/or the last wash section 202c), the AI based learning model may generate a hair stress level of a user, as represented by the sliding scale and corresponding indicator within the hair stress level section 206d. Generally, the indicator located on the sliding scale of the hair stress level section 206d may graphically illustrate the user's hair stress level to the user as between low hair stress and high hair stress. In the example aspect illustrated in FIG. 2, the user's hair stress level represented in the hair stress level section 206d indicates that the user's hair stress level is relatively low. The indicator may also provide a numerical representation of the user's hair stress level, and may indicate to a user that the user's hair stress level is, for example, a 95 out of a potential maximum score of 100. Of course, the scoring scale may include any suitable minimum and/or maximum values (e.g., 0 to 5, 1 to 6, 0 to 100, etc.). Regardless, it is to be understood that the hair stress level may be represented to a user as a graphical rendering (e.g., the sliding scale and indicator of the hair stress level section 206d), an alphanumerical value, a color value, and/or any other suitable representation or combinations thereof.

In addition, the user-specific data submitted by a user as inputs to the AI based learning model may include image data of the user. Specifically, in certain aspects, the image data may include one or more sebum images that define an amount of human sebum identifiable within the pixel data of the one or more sebum images. These sebum images, as described herein, may be captured in accordance with the tapping techniques previously mentioned and/or via the portable microscope device 112c4 of FIG. 1. Each sebum image may be used to train and/or execute the AI based learning model for use across a variety of different users having a variety of different scalp or hair region features. For example, as illustrated for image 114 in FIG. 1, the scalp or hair region of the user of this image comprises scalp and hair region features of the user's scalp that are identifiable with the pixel data of the image 114. These scalp and hair region features include, for example, white sebum residue and one or more lines/cracks of the scalp skin, which the AI based learning model may identify within the image 114, and may use to generate a scalp or hair prediction value (e.g., any one or more of the scalp quality score section 206a, the scalp turnover section 206b, the scalp stress level section 206c, and the hair stress level section 206d) and/or a user-specific treatment for the user represented in the image 114, as described herein.

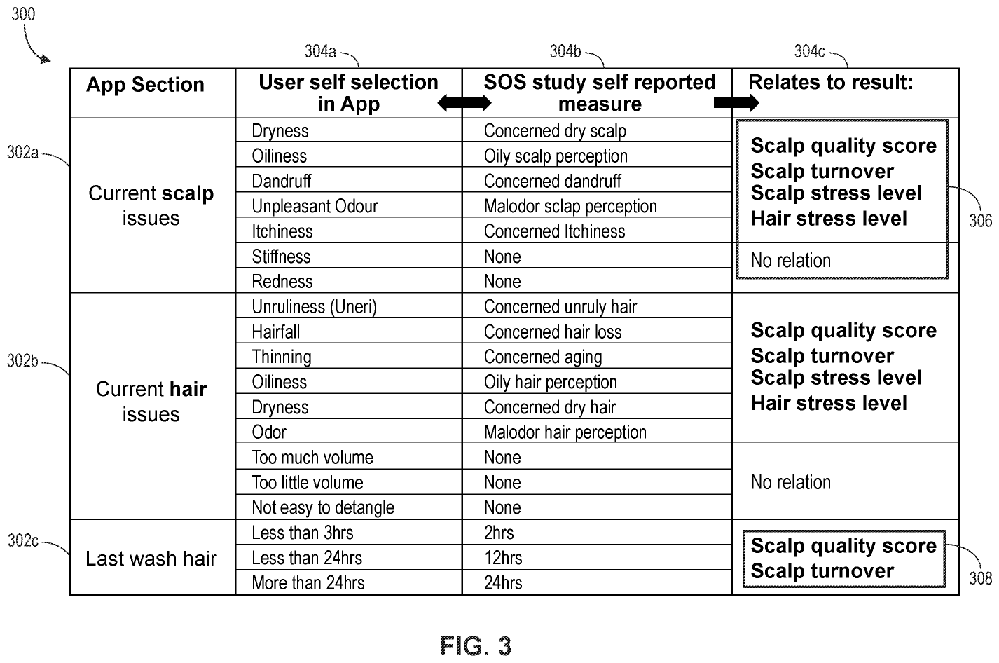

FIG. 3 illustrates an example correlation table 300 having scalp and hair factors correlating to outputs of the AI based learning model, in accordance with various aspects disclosed herein. Generally, the correlation table 300 provides an illustrative representation of the correlations drawn between the user-specific data (e.g., each of the issues/data included as part of the current scalp issues section 302a, the current hair issues section 302b, and the last wash hair section 302c) and the scalp or hair prediction values (e.g., represented by the values included in the result column 304c). The correlation table 300 includes each of the user-specific data types previously discussed (e.g., current scalp issues, current hair issues, and last wash data), and as represented in sections 302a, 302b, and 302c. While FIG. 3 illustrates three user-specific data types for user-specific data, including current scalp issues, current hair issues, and last wash data, it is to be understood that additional data types (e.g., such as user lifestyle/habits) are similarly contemplated herein.

Further, the correlation table 300 includes a user self-selection issue column 304a, a self-reported measure column 304b, and the result column 304c. Each column 304a, 304b, and 304c includes values that are correlated to values in other columns through a multiple correlation framework (e.g., as illustrated in FIG. 2), that is defined by a statistical analysis that is trained/utilized by the AI based learning model.

More specifically, the training the AI based learning model may include configuring a multivariate regression analysis using clinical data (e.g., data captured by the scalp or hair measurement device 111c4) to correlate each value/response included as part of the user-specific data to scalp and hair prediction values. As an example, the AI based learning model may comprise or utilize a multivariate regression analysis of the form:

P=M(V)+x₁O_{Hp}+x₂O_{Sp}+x₃M_{SP}+x₄M_{HP}+x₅C_{DA}+x₆C_{DS}+x₇C_{IS}+x₈C_{DH}+x₉C_{UH}+x₁₀C_{A}+x₁₁C_{HL} (1)

where P is a respective scalp or hair prediction value, M(V) is a matching formula configured to associate a particular weighted value with a user's input regarding the last time the user washed their hair, each of O_{HP}, O_{SP}, M_{SP}, M_{HP}, C_{DA}, C_{DS}, C_{IS}, C_{DH}, C_{UH}, C_{A}, and C_{HL} represent user-specific concerns/perceptions based on responses/inputs corresponding to the user-specific data values (e.g., listed in column 304a), and each xₙ (where n is a value between 1 and 11) is a weighting value corresponding to the related user-specific concerns/perceptions. Specifically, O_{HP} is the model value representing a user's oily hair perception and its corresponding impact on the scalp or hair prediction value. O_{SP} is the model value representing a user's oily scalp perception and its corresponding impact on the scalp or hair prediction value. M_{SP} is the model value representing a user's malodor scalp perception and its corresponding impact on the scalp or hair prediction value. M_{HP} is the model value representing a user's malodor hair perception and its corresponding impact on the scalp or hair prediction value. C_{DA} is the model value representing a user's dandruff concern and its corresponding impact on the scalp or hair prediction value. C_{DS} is the model value representing a user's dry scalp concern and its corresponding impact on the scalp or hair prediction value. C_{IS} is the model value representing a user's itchy scalp concern and its corresponding impact on the scalp or hair prediction value. C_{DH} is the model value representing a user's dry hair concern and its corresponding impact on the scalp or hair prediction value. C_{UH} is the model value representing a user's unruly hair concern and its corresponding impact on the scalp or hair prediction value. C_{A} is the model value representing a user's aging concern and its corresponding impact on the scalp or hair prediction value. C_{HL} is the model value representing a user's hair loss concern and its corresponding impact on the scalp or hair prediction value.

More generally, each of the user-specific concerns/perceptions may correspond to a binary (e.g., yes/no) response from the user related to a corresponding user-specific data value, and/or may correspond to a sliding scale value, an alphanumeric value, a multiple choice response (e.g., yes/no/maybe), and/or any other suitable response type or combinations thereof. Utilizing a regression model similar to the general model provided in equation (1), and as previously mentioned, the AI based learning model may achieve approximately 75% accuracy when generating scalp or hair prediction values for users based upon user-specific data, reflecting a substantial correlation between the AI based learning model and the user's actual scalp and hair condition that conventional techniques simply cannot not achieve.

For example, assume that the AI based learning model receives user input regarding each of the user-specific data represented in each of the sections 302a, 302b, 302c, and more particularly, in the user self-selection issue column 304a. Assume that the user indicates that they are concerned about scalp dryness (first entry in column 304a), the user last washed their hair less than 24 hours ago, and they have no concerns related to any of the other issues included in the user self-selection issue column 304a. In this scenario, the AI based learning model may determine (1) that the user is potentially concerned about having a dry scalp, as indicated in the corresponding first entry in the self-reported measure column 304b; (2) the user likely last washed their hair approximately 12 hours prior to providing the user input, as indicated in the second to last entry in the self-reported measure column 304b; and (3) that the user does not perceive and/or is not concerned with the other issues in column 304a and the corresponding concerns/perceptions in column 304b. Accordingly, the AI based learning model may correlate the user input to the values in the result column 304c by applying the regression model generally described in equation (1) to generate the user scalp or hair prediction values (e.g., the scalp quality score section 206a, the scalp turnover section 206b, the scalp stress level section 206c, and the hair stress level section 206d of FIG. 2).

The result column 304c generally includes representations of the relative strength of correlations between the values included in each of the user self-selection issue column 304a and the self-reported measure column 304b and the scalp or hair prediction values included in the result column 304c. For example, as illustrated in the current scalp issues result section 306, the values included in the corresponding sections of columns 304a and 304b most strongly correlate to the scalp quality score, and least strongly correlate to the hair stress level. In fact, two values (e.g., stiffness and redness) do not correlate to any of the scalp or hair prediction values included in the result column 304c. As another example, and as illustrated in the last wash hair result section 308, the values included in the corresponding sections of columns 304a and 304b most strongly correlate to the scalp quality score, correlate less strongly to scalp turnover, and do not correlate at all to the scalp stress level or the hair stress level.

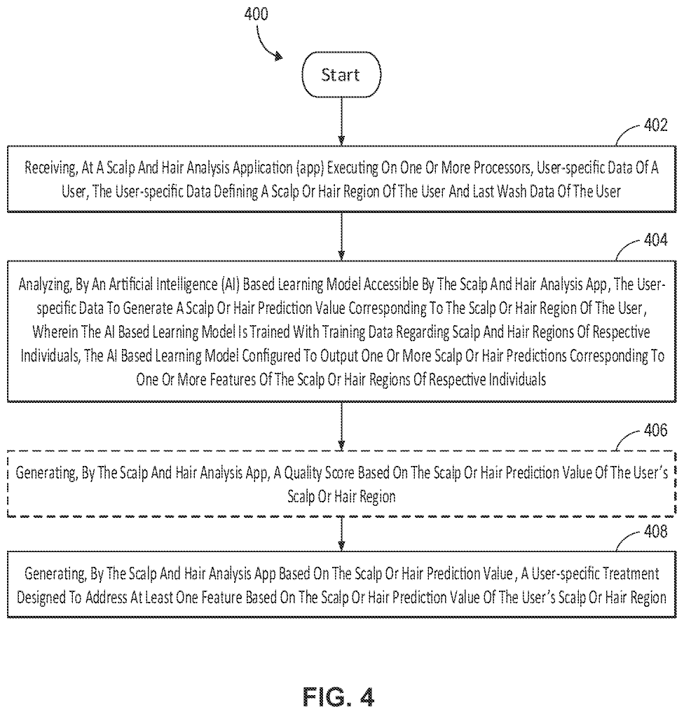

FIG. 4 illustrates an AI based method 400 for analyzing user-specific skin or hair data to predict user-specific skin or hair conditions, in accordance with various aspects disclosed herein. The user-specific data, as used with the method 400, and more generally as described herein, is user responses/inputs received by a user computing device (e.g., user computing device 111c1). In some aspects, the user-specific data may comprise or refer to a plurality of responses/inputs such as a plurality of user responses collected by the user computing device while executing the scalp and hair analysis application (app), described herein.

At block 402, the method 400 comprises receiving, at a scalp and hair analysis application (app) executing on one or more processors (e.g., one or more processor(s) 104 of server(s) 102 and/or processors of a computer user device, such as a mobile device), user-specific data of a user. The user-specific data may define a scalp or hair region of the user and last wash data of the user. Generally, the user-specific data may comprise non-image data, such as user responses/inputs to a questionnaire presented as part of the execution of the scalp and hair analysis app. The scalp or hair region defined by the user-specific data may correspond to one of (1) a scalp region of the user, (2) a hair region of the user, and/or any other suitable scalp or hair region of the user or combinations thereof.

However, in certain aspects, the user-specific data may comprise both image data and non-image data, wherein the image data may be a digital image as captured by an imaging device (e.g., an imaging device of user computing device 111c1 or 112c4). In these aspects, the image data may comprise pixel data of at least a portion of a scalp or hair region of the user. Particularly, in certain aspects, the scalp or hair region of the user may include at least one of (i) a frontal scalp region, (ii) a frontal hair region, (iii) a mid-center scalp region, (iv) a mid-center hair region, (v) a custom defined scalp region, (vi) a custom defined hair region, (vii) a forehead region, and/or other suitable scalp or hair regions or combinations thereof.

In certain aspects, the one or more processors may comprise a processor of a mobile device, which may include at least one of a handheld device (e.g., user computing device 111c1) and/or a scalp or hair measurement device (e.g., scalp or hair measurement device 111c4). Accordingly, in these aspects, the handheld device and/or the scalp or hair measurement device may independently or collectively receive the user-specific data of the user. For example, if the handheld device executes the scalp and hair analysis app, the handheld device may receive user input to the questionnaire presented as part of the scalp and hair analysis app execution. Additionally, the user may apply the scalp or hair measurement device to the user's scalp or hair region to receive sebum data associated with the user. The handheld device and/or the scalp or hair measurement device may receive the user inputs and the sebum data (collectively, the user-specific data) to process/analyze the user-specific data, in accordance with the actions of the method 400 described herein.

Similarly, in certain aspects, the one or more processors may comprise a processor of a mobile device, which may include at least one of a handheld device (e.g., user computing device 111c1) and/or a portable microscope (e.g., portable microscope device 112c4). Accordingly, in these aspects, the imaging device may comprise the portable microscope, and the mobile device may execute the scalp and hair analysis app. For example, if the imaging device is a portable microscope (e.g., portable microscope device 112c4), the user may capture images of the user's scalp or hair region using the camera of the portable microscope, and the portable microscope may process/analyze the captured images using the one or more processors of the portable microscope and/or may transmit the captured images to a connected mobile device (e.g., user computing device 112c1) for processing/analysis, in accordance with the actions of the method 400 described herein.

At block 404, the method 400 comprises analyzing, by an AI based learning model (e.g., AI based learning model 108) accessible by the scalp and hair analysis app, the user-specific data to generate a scalp or hair prediction value corresponding to the scalp or hair region of the user. Particularly, the scalp or hair prediction value may correspond to one or more features of the scalp or hair region of the user. In certain aspects, the scalp or hair prediction value comprises a sebum prediction value that may correspond to a predicted sebum level associated with the scalp or hair region of the user.

An AI based learning model (e.g., AI based learning model 108) as referred to herein in various aspects, is trained with training data regarding scalp and hair regions of respective individuals. The AI based learning model is configured to, or is otherwise operable to, output one or more scalp or hair predictions corresponding to one or more features of the scalp or hair regions of respective individuals. The training data comprises data (e.g., clinical data) generated with a scalp or hair measurement device (e.g., scalp or hair measurement device 111c4) configured to determine the one or more features of the scalp or hair regions. In certain aspects, the scalp or hair measurement device is configured to determine a sebum level of a skin surface of the user.

Further, the training data regarding scalp and hair regions of respective individuals is selected from one or more values corresponding to last wash data of the respective individuals and at least one of: one or more of scalp factors, one or more hair factors, and/or wash frequency. Thus, each instance of training data must include at least a last wash data of a respective individual in order to train the AI based learning model because, as previously mentioned, the scalp or hair predictions output by the AI based learning model are influenced significantly by the user's last wash data. In various aspects, the one or more scalp factors comprise scalp dryness, scalp oiliness, dandruff, stiffness, redness, unpleasant odor, or itchiness. In various aspects, the one or more hair factors comprise unruliness, hair fall, hair volume, thinning, detangling, hair oiliness, dryness, or hair odor.

For example, a first set of training data corresponding to a first respective individual may include last wash data indicating that the first respective individual last washed their hair less than 3 hours before submitting their responses/inputs, and may further indicate that the first respective individual is concerned about scalp dryness. Further in this example, a second set of training data corresponding to a second respective individual may include last wash data indicating that the second respective individual last washed their hair more than 24 hours before submitting their responses/inputs, and may further indicate that the second respective individual is concerned about hair thinning. Finally, in this example, a third set of data corresponding to a third respective individual may not include last wash data, and may indicate that the third respective individual is concerned about scalp dandruff and hair oiliness. In this example, the AI based learning model may be trained with the first and second set of training data, but not the third set of data because the first and second set of training data include last wash data and the third set of data does not.

Moreover, in various aspects, the training data comprises image data and non-image data of the respective individuals, and the user-specific data comprises image data and non-image data of the user. In these aspects, the image data of the training data and the image data of the user-specific data each comprise one or more sebum images defining an amount of human sebum identifiable within pixel data of the one or more sebum images.

As previously mentioned, the AI based learning model, as described herein (e.g. AI based learning model 108), may be trained using a supervised machine learning program or algorithm, such as multivariate regression analysis. Generally, machine learning may involve identifying and recognizing patterns in existing data (such as generating scalp or hair predictions corresponding to one or more features of the scalp or hair regions of respective individuals) in order to facilitate making predictions or identification for subsequent data (such as using the model on new user-specific data in order to determine or generate a scalp or hair prediction value corresponding to the scalp or hair region of a user and/or a user-specific treatment to address at least one feature based on the scalp or hair prediction value). Machine learning model(s), such as the AI based learning model described herein for some aspects, may be created and trained based upon example data (e.g., “training data” and related user-specific data) inputs or data (which may be termed “features” and “labels”) in order to make valid and reliable predictions for new inputs, such as testing level or production level data or inputs.

In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

However, while described herein as being trained using a supervised learning technique (e.g., multivariate regression analysis), in certain aspects, the AI based learning model may be trained using multiple supervised machine learning techniques, and may additionally or alternatively be trained using one or more unsupervised machine learning techniques. In unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated.

For example, in certain aspects, the AI based learning model may employ a neural network, which may be a convolutional neural network, a deep learning neural network, or a combined learning module or program that learns in two or more features or feature datasets (e.g., user-specific data) in particular areas of interest. The machine learning programs or algorithms may also include natural language processing, semantic analysis, automatic reasoning, support vector machine (SVM) analysis, decision tree analysis, random forest analysis, K-Nearest neighbor analysis, naïve B ayes analysis, clustering, reinforcement learning, and/or other machine learning algorithms and/or techniques. In some aspects, the artificial intelligence and/or machine learning based algorithms may be included as a library or package executed on the server(s) 102. For example, libraries may include the TENSORFLOW based library, the PYTORCH library, and/or the SCIKIT-LEARN Python library.

Regardless, training the AI based learning model may also comprise retraining, relearning, or otherwise updating models with new, or different, information, which may include information received, ingested, generated, or otherwise used over time. Moreover, in various aspects, the AI based learning model (e.g., AI based learning model 108) may be trained, by one or more processors (e.g., one or more processor(s) 104 of server(s) 102 and/or processors of a computer user device, such as a mobile device) with the pixel data of a plurality of training images (e.g., image 114) of the scalp or hair regions of respective individuals. In these aspects, the AI based learning model (e.g., AI based learning model 108) may additionally be configured to generate one or more scalp or hair predictions corresponding to one or more features of the scalp or hair regions of each respective individual in each of the plurality of training images.

At optional block 406, the method 400 comprises generating, by the scalp and hair analysis app, a quality score based on the scalp or hair prediction value of the user's scalp or hair region. The quality score is generated or designed to indicate a quality (e.g., represented by the scalp quality score section 206a, of FIG. 2) of the user's scalp or hair region defined by the user-specific data. In various aspects, computing instructions of the hair and scalp analysis app when executed by one or more processors, may cause the one or more processors to generate the quality score as determined based on the scalp or hair prediction value of the user's scalp or hair region. The quality score may include any suitable scoring system/representation.

For example, in these aspects and as illustrated in FIG. 2, the quality score may include a graphical score (e.g., graphical score 206a1) that may indicate to a user that the user's scalp quality score is, for example, a 3.5 out of a potential maximum score of 4. The quality score may further include a quality score description (e.g., quality score description 206a20 that may include a description of, for example, a predominant scalp/hair factor and/or last wash data leading to a reduced score, endogenous/exogenous factors causing scalp and/or hair issues, and/or any other information or combinations thereof. Moreover, the quality score may include an average/sum value corresponding to the respective weighted values associated with the user-specific data analyzed and correlated to the quality score, as part of the AI based learning model (e.g., AI based learning model 108).

At block 408, the method 400 comprises generating, by the scalp and hair analysis app based on the scalp or hair prediction value, a user-specific treatment that is designed to address at least one feature based on the scalp or hair prediction value of the user's scalp or hair region. In various aspects, the user-specific treatment is displayed on the display screen of a computing device (e.g., user computing device 111c1) to instruct the user regarding how to treat the at least one feature based on the scalp or hair prediction value of the user's scalp or hair region.

The user-specific treatment may be generated by a user computing device (e.g., user computing device 111c1) and/or by a server (e.g., server(s) 102). For example, in some aspects the server(s) 102, as described herein for FIG. 1, may analyze user-specific data remote from a user computing device to determine a scalp or hair prediction value corresponding to the scalp or hair region of the user, a quality score, and/or the user-specific treatment designed to address at least one feature based on the scalp or hair prediction value of the user's scalp or hair region. For example, in such aspects the server or a cloud-based computing platform (e.g., server(s) 102) receives, across computer network 120, the user-specific data defining a scalp or hair region of the user comprising (1) last wash data of the user, and (2) at least one of: one or more scalp factors of the user, one or more hair factors of the user, or a wash frequency of the user. The server or a cloud-based computing platform may then execute the AI based learning model (e.g., AI based learning model 108) and generate, based on output of the AI based learning model, the scalp or hair prediction value, the quality score, and/or the user-specific treatment. The server or a cloud-based computing platform may then transmit, via the computer network (e.g., computer network 120), the scalp or hair prediction value, the quality score, and/or the user-specific treatment to the user computing device for rendering on the display screen of the user computing device. For example, and in various aspects, the scalp or hair prediction value, the quality score, and/or the user-specific treatment may be rendered on the display screen of the user computing device in real-time or near-real time, during, or after receiving, the user-specific data defining the scalp or hair region of the user and the last wash data of the user.

As an example, in various aspects, the user-specific treatment may include a recommended wash frequency specific to the user. The recommended wash frequency may comprise a number of times to wash, one or more times or periods over a day, week, etc. to wash, suggestions as to how to wash, etc. Moreover, in various aspects, the user-specific treatment may comprise a textually-based treatment, a visual/image based treatment, and/or a virtual rendering of the user's scalp or hair region, e.g., displayed on the display screen of a user computing device (e.g., user computing device 111c1). Such user-specific treatment may include a graphical representation of the user's scalp or hair region as annotated with one or more graphics or textual renderings corresponding to user-specific features (e.g., excessive scalp sebum, dandruff, dryness, etc.).

Further, in certain aspects, the scalp and hair analysis app may receive an image of the user, and the image may depict the scalp or hair region of the user. In these aspects, the scalp and hair analysis app may generate a photorealistic representation of the user after virtual application of the user-specific treatment to the scalp or hair region of the user. Further, the scalp and hair analysis app may generate the photorealistic representation by manipulating one or more pixels of the image of the user based on the scalp or hair prediction value. For example, the scalp and hair analysis app may graphically render the user-specific treatment for display to a user, and the user-specific treatment may include a treatment option to increase hair/scalp washing frequency to reduce scalp sebum build-up that the AI based learning model determined is present in the user's scalp or hair region based on the user-specific data and last wash data. In this example, the scalp and hair analysis app may generate a photorealistic representation of the user's scalp or hair region without scalp sebum (or a reduced amount) by manipulating the pixel values of one or more pixels of the image (e.g., updating, smoothing, changing colors) of the user to alter the pixel values of pixels identified as containing pixel data representative of scalp sebum present on the user's scalp or hair region to pixel values representative of the user's scalp skin or hair follicles in the user's scalp or hair region. For example, in some aspects, the graphical representation of the user's scalp or hair region 506 is the photorealistic representation of the user.

In additional aspects, the user-specific treatment may comprise a product recommendation for a manufactured product. Additionally, or alternatively, in some aspects, the user-specific treatment may be displayed on the display screen of a computing device (e.g., user computing device 111c1) with instructions (e.g., a message) for treating, with the manufactured product, the at least one feature based on the scalp or hair prediction value of the user's scalp or hair region. In still further aspects, computing instructions, executing on processor(s) of either a user computing device (e.g., user computing device 111c1) and/or server(s) may initiate, based on the user-specific treatment, the manufactured product for shipment to the user. With regard to manufactured product recommendations, in some aspects, one or more processors (e.g., server(s) 102 and/or a user computing device, such as user computing device 111c1) may generate and render a modified image, as previously described, based on how the user's scalp or hair regions are predicted to appear after treating the at least one feature with the manufactured product.

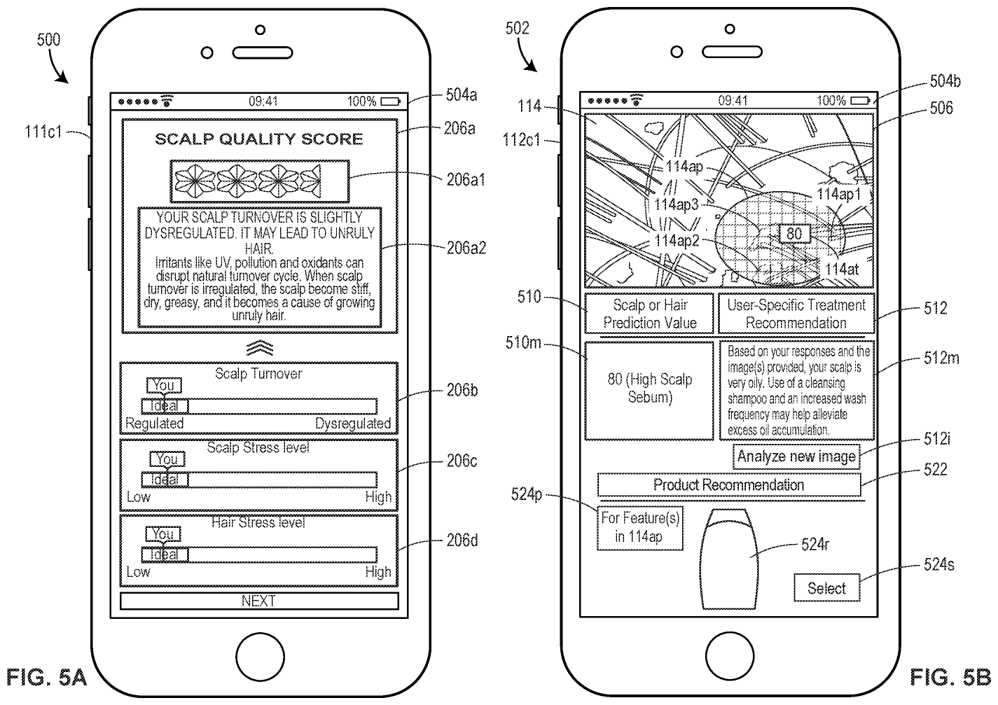

FIG. 5A illustrates an example user interface 504a as rendered on a display screen 500 of a user computing device (e.g., user computing device 111c1) in accordance with various aspects disclosed herein. For example, as shown in the example of FIG. 5A, the user interface 504a may be implemented or rendered via an application (app) executing on user computing device 111c1. For example, as shown in the example of FIG. 5A, user interface 504a may be implemented or rendered via a native app executing on user computing device 111c1. In the example of FIG. 5A, user computing device 111c1 is a user computing device as described for FIG. 1, e.g., where 111c1 is illustrated as an APPLE iPhone that implements the APPLE iOS operating system and that has display screen 500. User computing device 111c1 may execute one or more native applications (apps) on its operating system, including, for example, the scalp and hair analysis app, as described herein. Such native apps may be implemented or coded (e.g., as computing instructions) in a computing language (e.g., SWIFT) executable by the user computing device operating system (e.g., APPLE iOS) by the processor of user computing device 111c1.

Additionally, or alternatively, user interface 504a may be implemented or rendered via a web interface, such as via a web browser application, e.g., Safari and/or Google Chrome app(s), or other such web browser or the like.

As shown in the example of FIG. 5A, user interface 504a comprises a graphical representation of the scalp or hair prediction values, including the scalp quality score section 206a, the graphical score 206a1, the scalp quality score description 206a2, the scalp turnover section 206b, the scalp stress level section 206c, and the hair stress level section 206d of FIG. 2. Accordingly, the scalp and hair analysis app may directly convey each of the scalp or hair prediction values to a user by rendering the scalp or hair prediction values on the user interface 504a. In certain aspects, the scalp and hair analysis app may render the user interface 504a first in a series of graphical displays intended to provide the user with a comprehensive evaluation of the user's scalp or hair region defined by the user-specific data and the last wash data.

For example, FIG. 5B illustrates another example user interface 504b as rendered on a display screen 502 of a user computing device (e.g., user computing device 111c1), and in certain aspects, the user interface 504b may be a subsequent graphical rendering to the user interface 504a of FIG. 5A. The user interface 504b comprises a graphical representation (e.g., of image 114) of a user's scalp or hair region 506. The image 114 may comprise an image of the user (or graphical representation 506 thereof) comprising pixel data (e.g., pixel data 114ap) of at least a portion of the scalp or hair region of the user, as described herein. In the example of FIG. 5B, the graphical representation (e.g., of image 114) of the user's scalp or hair region 506 is annotated with one or more graphics (e.g., areas of pixel data 114ap) or textual rendering(s) (e.g., text 114at) corresponding to various features identifiable within the pixel data comprising a portion of the scalp or hair region of the user. For example, the area of pixel data 114ap may be annotated or overlaid on top of the image of the user (e.g., image 114) to highlight the area or feature(s) identified within the pixel data (e.g., feature data and/or raw pixel data) by the AI based learning model (e.g., AI based learning model 108). In the example of FIG. 5B, the area of pixel data 114ap indicates features, as defined in pixel data 114ap, including scalp sebum (e.g., for pixels 114ap1-3), and may indicate other features shown in area of pixel data 114ap (e.g., scalp dryness, scalp oiliness, scalp dandruff, hair unruliness, hair dryness, etc.), as described herein. In various aspects, the pixels identified as the specific features (e.g., pixels 114ap1-3), may be highlighted or otherwise annotated when rendered on display screen 502.

The textual rendering (e.g., text 114at) shows a user-specific attribute or feature (e.g., 80 for pixels 114ap1-3) which may indicate that the user has a high scalp quality score (of 80) for scalp sebum. The 80 score indicates that the user has a high amount of sebum present on the user's scalp or hair region (and therefore likely the user's entire scalp), such that the user would likely benefit from washing their scalp with a cleansing shampoo and increasing their washing frequency to improve their scalp health/quality/condition (e.g., reduce the amount of scalp sebum). It is to be understood that other textual rendering types or values are contemplated herein, where textual rendering types or values may be rendered, for example, such as a scalp quality score, a scalp turnover score, a scalp stress level score, a hair stress level score, or the like. Additionally, or alternatively, color values may be used and/or overlaid on a graphical representation (e.g., graphical representation of the user's scalp or hair region 506) shown on user interface 504b to indicate a degree or quality of a given score, e.g., a high score of 80 or a low score of 5. The scores may be provided as raw scores, absolute scores, percentage based scores, and/or any other suitable presentation style. Additionally, or alternatively, such scores may be presented with textual or graphical indicators indicating whether or not a score is representative of positive results (good scalp washing frequency), negative results (poor scalp washing frequency), or acceptable results (average or acceptable scalp washing frequencies).

User interface 502 may also include or render a scalp or hair prediction value 510. In the aspect of FIG. 5B, the scalp or hair prediction value 510 comprises a message 510m to the user designed to indicate the scalp or hair prediction value to the user, along with a brief description of any reasons resulting in the scalp or hair prediction value. As shown in the example of FIG. 5B, the message 510m indicates to a user that the scalp or hair prediction value is “80” and further indicates to the user that the scalp or hair prediction value results from the scalp or hair region of the user containing “high scalp sebum.”

User interface 504b may also include or render a user-specific treatment recommendation 512. In the aspect of FIG. 5B, user-specific treatment recommendation 512 comprises a message 512m to the user designed to address at least one feature identifiable within the user-specific data defining the scalp or hair region of the user and the last wash data of the user. As shown in the example of FIG. 5B, message 512m recommends to the user to wash their scalp at a higher washing frequency to improve their scalp health/quality/condition by reducing excess sebum build-up.

Message 512m further recommends use of a cleansing shampoo to help reduce the excess sebum build-up. The cleansing shampoo recommendation can be made based on the high scalp quality score for scalp sebum (e.g., 80) suggesting that the image of the user depicts a high amount of scalp sebum, where the cleansing shampoo product is designed to address scalp sebum detected or classified in the pixel data of image 114 or otherwise predicted based on the user-specific data and last wash data of the user. The product recommendation can be correlated to the identified feature within the user-specific data and/or the pixel data, and the user computing device 111c1 and/or server(s) 102 can be instructed to output the product recommendation when the feature (e.g., excessive scalp (or hair) sebum) is identified.

The user interface 504b may also include or render a section for a product recommendation 522 for a manufactured product 524r (e.g., cleansing shampoo, as described above). The product recommendation 522 may correspond to the user-specific treatment recommendation 512, as described above. For example, in the example of FIG. 5B, the user-specific treatment recommendation 512 may be displayed on the display screen 502 of user computing device 111c1 with instructions (e.g., message 512m) for treating, with the manufactured product (manufactured product 524r (e.g., cleansing shampoo)) at least one feature (e.g., high scalp quality score of 80 related to scalp sebum at pixels 114ap1-3) predicted and/or identifiable based on the user-specific data and last wash data and/or, in certain aspects, the pixel data (e.g., pixel data 114ap) comprising pixel data of at least a portion of a scalp or hair region of the user. The features predicted or identified are indicated and annotated (524p) on the user interface 504b.

As shown in FIG. 5B, the user interface 504b recommends a product (e.g., manufactured product 524r (e.g., cleansing shampoo)) based on the user-specific treatment recommendation 512. In the example of FIG. 5B, the output or analysis of the user-specific data and the image(s) (e.g. image 114) by the AI based learning model (e.g., AI based learning model 108), e.g., scalp or hair prediction value 510 and/or its related values (e.g., 80 scalp sebum quality score) or related pixel data (e.g., 114ap1, 114ap2, and/or 114ap3), and/or the user-specific treatment recommendation 512, may be used to generate or identify recommendations for corresponding product(s). Such recommendations may include products such as shampoo, conditioner, hair gel, moisturizing treatments, and the like to address the user-specific issue as detected or predicted from the user-specific data and last wash data and/or, in certain aspects, within the pixel data by the AI based learning model (e.g., AI based learning model 108).

User interface 504b may further include a selectable UI button 524s to allow the user (e.g., the user of image 114) to select for purchase or shipment the corresponding product (e.g., manufactured product 524r). In some aspects, selection of selectable UI button 524s may cause the recommended product(s) to be shipped to the user and/or may notify a third party that the individual is interested in the product(s). For example, either user computing device 111c1 and/or the server(s) 102 may initiate, based on the scalp or hair prediction value 510 and/or the user-specific treatment recommendation 512, the manufactured product 524r (e.g., cleansing shampoo) for shipment to the user. In such aspects, the product may be packaged and shipped to the user.

In various aspects, a graphical representation (e.g., graphical representation of the user's scalp or hair region 506), with graphical annotations (e.g., area of pixel data 114ap), textual annotations (e.g., text 114at), and the scalp or hair prediction value 510 and the user-specific treatment recommendation 512 may be transmitted, via the computer network (e.g., from a server 102 and/or one or more processors) to the user computing device 111c1, for rendering on the display screen 500, 502. In other aspects, no transmission to the server of the user's specific image occurs, where the scalp or hair prediction value 510 and the user-specific treatment recommendation 512 (and/or product specific recommendation) may instead be generated locally, by the AI based learning model (e.g., AI based learning model 108) executing and/or implemented on the user's mobile device (e.g., user computing device 111c1) and rendered, by a processor of the mobile device, on display screen 500, 502 of the mobile device (e.g., user computing device 111c1).

In some aspects, any one or more of graphical representations (e.g., graphical representation of the user's scalp or hair region 506), with graphical annotations (e.g., area of pixel data 114ap), textual annotations (e.g., text 114at), scalp or hair prediction value 510, user-specific treatment recommendation 512, and/or product recommendation 522 may be rendered (e.g., rendered locally on display screen 500, 502) in real-time or near-real time during or after receiving, the user-specific data and last wash data and/or, in certain aspects, the image having the scalp or hair region of the user. In aspects where the user-specific data and last wash data and the image is analyzed by a server(s) 102, the user-specific data and last wash data and the image may be transmitted and analyzed in real-time or near real-time by the server(s) 102.

In some aspects, the user may provide new user-specific data, new last wash data, and/or a new image that may be transmitted to the server(s) 102 for updating, retraining, or reanalyzing by the AI based learning model 108. In other aspects, new user-specific data, new last wash data, and/or a new image may be locally received on computing device 111c1 and analyzed, by the AI based learning model 108, on the computing device 111c1.

In addition, as shown in the example of FIG. 5B, the user may select selectable button 512i for reanalyzing (e.g., either locally at computing device 111c1 or remotely at the server(s) 102) new user-specific data, new last wash data, and/or a new image. Selectable button 512i may cause the user interface 504b to prompt the user to input/attach for analyzing new user-specific data, new last wash data, and/or a new image. The server(s) 102 and/or a user computing device such as user computing device 111c1 may receive the new user-specific data, new last wash data, and/or a new image comprising data that defines a scalp or hair region of the user. The new user-specific data, new last wash data, and/or new image may be received/captured by the user computing device. A new image (e.g., similar to image 114) may comprise pixel data of a portion of a scalp or hair region of the user. The AI based learning model (e.g., AI based learning model 108), executing on the memory of the computing device (e.g., server(s) 102), may analyze the new user-specific data, new last wash data, and/or new image received/captured by the user computing device to generate a new scalp or hair prediction value. The computing device (e.g., server(s) 102) may generate, a new scalp or hair prediction value based on a comparison of the new user-specific data and the user-specific data, the new last wash data and the last wash data, and/or the new image and the image. For example, the new scalp or hair prediction value may include a new graphical representation including graphics and/or text (e.g., showing a new quality score value, e.g., 1, after the user washed their hair). The new scalp or hair prediction value may include additional quality scores, e.g., that the user has successfully washed their hair to reduce scalp dandruff and/or hair oiliness as detected with the new user-specific data, the new last wash data, and/or the new image. A comment may include that the user needs to correct additional features detected within the new user-specific data, the new last wash data, and/or the new image, e.g., hair dryness, by applying an additional product, e.g., moisturizing shampoo or coconut oil.

In various aspects, the new scalp or hair prediction value and/or a new user-specific treatment recommendation may be transmitted via the computer network, from server(s) 102, to the user computing device of the user for rendering on the display screen 502 of the user computing device (e.g., user computing device 111c1).

In other aspects, no transmission to the server of the user's new user-specific data, new last wash data, and/or new image occurs, where the new u scalp or hair prediction value and/or the new user-specific treatment recommendation (and/or product specific recommendation) may instead be generated locally, by the AI based learning model (e.g., AI based learning model 108) executing and/or implemented on the user's mobile device (e.g., user computing device 111c1) and rendered, by a processor of the mobile device, on a display screen of the mobile device (e.g., user computing device 111c1).

Of course, it is to be understood that any of the graphical/textual renderings present on user interfaces 504a, 504b may be rendered on either of user interfaces 504a, 504b. For example, the scalp quality score section 206a present in the user interface 504a may be rendered as part of the display in user interface 504b. Similarly, the scalp or hair prediction value 510 and the user-specific treatment recommendation 512 and corresponding messages 510m, 512m may be rendered as part of the display in user interface 504a.

### ASPECTS OF THE DISCLOSURE

The following aspects are provided as examples in accordance with the disclosure herein and are not intended to limit the scope of the disclosure.

1. An artificial intelligence (AI) based system configured to analyze user-specific skin or hair data to predict user-specific skin or hair conditions, the AI based system comprising: one or more processors; an scalp and hair analysis application (app) comprising computing instructions configured to execute on the one or more processors; and an AI based learning model, accessible by the scalp and hair analysis app, and trained with training data regarding scalp and hair regions of respective individuals, the AI based learning model configured to output one or more scalp or hair predictions corresponding to one or more features of the scalp or hair regions of respective individuals, wherein the training data regarding scalp and hair regions of respective individuals is selected from one or more values corresponding to last wash data of the respective individuals and at least one of: one or more of scalp factors, one or more hair factors, or wash frequency, wherein the training data comprises data generated with a scalp or hair measurement device configured to determine the one or more features of the scalp or hair regions, wherein the computing instructions of the scalp and hair analysis app when executed by the one or more processors, cause the one or more processors to: receive user-specific data of a user, the user-specific data defining a scalp or hair region of the user comprising (1) last wash data of the user, and (2) at least one of: one or more scalp factors of the user, one or more hair factors of the user, or a wash frequency of the user, analyze, by the AI based learning model, the user-specific data to generate a scalp or hair prediction value corresponding to the scalp or hair region of the user, and generate, based on the scalp or hair prediction value, a user-specific treatment designed to address at least one feature based on the scalp or hair prediction value of the user's scalp or hair region.

2. The AI based system of aspect 1, wherein the one or more scalp factors comprise scalp dryness, scalp oiliness, dandruff, stiffness, redness, unpleasant odor, or itchiness.

3. The AI based system of aspect 2, wherein the scalp or hair prediction value comprises a sebum prediction value.

4. The AI based system of any one of aspects 1-3, wherein the scalp or hair region of the user corresponds to one of: (1) a scalp region of the user; or (2) a hair region of the user.

5. The AI based system of any one of aspects 1-4, wherein the one or more hair factors comprise unruliness, hair fall, hair volume, thinning, detangling, hair oiliness, dryness, or hair odor.

6. The AI based system of any one of aspects 1-5, wherein the computing instructions of the scalp and hair analysis app when executed by the one or more processors, further cause the one or more processors to: generate a quality score based on the scalp or hair prediction value of the user's scalp or hair region.

7. The AI based system of any one of aspects 1-6, wherein the training data comprises image data and non-image data of the respective individuals, and wherein the user-specific data comprises image data and non-image data of the user.

8. The AI based system of aspect 7, wherein the image data of the training data and the image data of the user-specific data each comprise one or more sebum images defining an amount of human sebum identifiable within pixel data of the one or more sebum images.

9. The AI based system of any one of aspects 1-8, wherein the computing instructions of the scalp or hair analysis app when executed by the one or more processors, further cause the one or more processors to: receive an image of the user, the image depicting the scalp or hair region of the user, and generate a photorealistic representation of the user after virtual application of the user-specific treatment to the scalp or hair region of the user, the photorealistic representation generated by manipulating one or more pixels of the image of the user based on the scalp or hair prediction value.

10. The AI based system of any one of aspects 1-9, wherein the scalp or hair measurement device is configured to determine a sebum level of a skin surface of the user.

11. An artificial intelligence (AI) based method for analyzing user-specific skin or hair data to predict user-specific skin or hair conditions, the AI based method comprising: receiving, at a scalp and hair analysis application (app) executing on one or more processors, user-specific data of a user, the user-specific data defining a scalp or hair region of the user comprising (1) last wash data of the user, and (2) at least one of: one or more scalp factors of the user, one or more hair factors of the user, or a wash frequency of the user; analyzing, by an artificial intelligence (AI) based learning model accessible by the scalp and hair analysis app, the user-specific data to generate a scalp or hair prediction value corresponding to the scalp or hair region of the user, wherein the AI based learning model is trained with training data regarding scalp and hair regions of respective individuals and is configured to output one or more scalp or hair predictions corresponding to one or more features of the scalp or hair regions of respective individuals, wherein the training data regarding scalp and hair regions of respective individuals is selected from one or more values corresponding to last wash data of the respective individuals and at least one of: one or more of scalp factors, one or more hair factors, or wash frequency, wherein the training data comprises data generated with a scalp or hair measurement device configured to determine the one or more features of the scalp or hair regions; and generating, by the scalp and hair analysis app based on the scalp or hair prediction value, a user-specific treatment designed to address at least one feature based on the scalp or hair prediction value of the user's scalp or hair region.

12. The AI based method of aspect 11, wherein the one or more scalp factors comprise scalp dryness, scalp oiliness, dandruff, stiffness, redness, unpleasant odor, or itchiness.

13. The AI based method of aspect 12, wherein the scalp or hair prediction value comprises a sebum prediction value.

14. The AI based method of any one of aspects 11-13, wherein the scalp or hair region of the user corresponds to one of: (1) a scalp region of the user; or (2) a hair region of the user.

15. The AI based method of any one of aspects 11-14, wherein the one or more hair factors comprise unruliness, hair fall, hair volume, thinning, detangling, hair oiliness, dryness, or hair odor.

16. The AI based method of any one of aspects 11-15, the method further comprising: generating, by the scalp and hair analysis app, a quality score based on the scalp or hair prediction value of the user's scalp or hair region.

17. The AI based method of any one of aspects 11-16, wherein the training data comprises image data and non-image data of the respective individuals, and wherein the user-specific data comprises image data and non-image data of the user.

18. The AI based method of any one of aspects 11-17, wherein the image data of the training data and the image data of the user-specific data each comprise one or more sebum images defining an amount of human sebum identifiable within pixel data of the one or more sebum images.

19. The AI based method of any one of aspects 11-18, the method further comprising: receiving, at the scalp and hair analysis app, an image of the user, the image depicting the scalp or hair region of the user; and generating, by the scalp and hair analysis app, a photorealistic representation of the user after virtual application of the user-specific treatment to the scalp or hair region of the user, the photorealistic representation generated by manipulating one or more pixels of the image of the user based on the scalp or hair prediction value.

20. The AI based method of any one of aspects 11-19, wherein the scalp or hair measurement device is configured to determine a sebum level of a skin surface of the user.

21. A tangible, non-transitory computer-readable medium storing instructions for analyzing user-specific skin or hair data to predict user-specific skin or hair conditions, that when executed by one or more processors cause the one or more processors to: receive, at a scalp and hair analysis application (app) executing on one or more processors, user-specific data of a user, the user-specific data defining a scalp or hair region of the user comprising (1) last wash data of the user, and (2) at least one of: one or more scalp factors of the user, one or more hair factors of the user, or a wash frequency of the user; analyze, by an artificial intelligence (AI) based learning model accessible by the scalp and hair analysis app, the user-specific data to generate a scalp or hair prediction value corresponding to the scalp or hair region of the user, wherein the AI based learning model is trained with training data regarding scalp and hair regions of respective individuals and is configured to output one or more scalp or hair predictions corresponding to one or more features of the scalp or hair regions of respective individuals, wherein the training data regarding scalp and hair regions of respective individuals is selected from one or more values corresponding to last wash data of the respective individuals and at least one of: one or more of scalp factors, one or more hair factors, or wash frequency, wherein the training data comprises data generated with a scalp or hair measurement device configured to determine the one or more features of the scalp or hair regions; and generate, by the scalp and hair analysis app based on the scalp or hair prediction value, a user-specific treatment designed to address at least one feature based on the scalp or hair prediction value of the user's scalp or hair region.

### ADDITIONAL CONSIDERATIONS

Although the disclosure herein sets forth a detailed description of numerous different aspects, it should be understood that the legal scope of the description is defined by the words of the claims set forth at the end of this patent and equivalents. The detailed description is to be construed as exemplary only and does not describe every possible aspect since describing every possible aspect would be impractical. Numerous alternative aspects may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain aspects are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example aspects, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example aspects, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example aspects, the processor or processors may be located in a single location, while in other aspects the processors may be distributed across a number of locations.

The performance of certain of the operations may be distributed among the one or more processors, not only residing within a single machine, but deployed across a number of machines. In some example aspects, the one or more processors or processor-implemented modules may be located in a single geographic location (e.g., within a home environment, an office environment, or a server farm). In other aspects, the one or more processors or processor-implemented modules may be distributed across a number of geographic locations.

This detailed description is to be construed as exemplary only and does not describe every possible aspect, as describing every possible aspect would be impractical, if not impossible. A person of ordinary skill in the art may implement numerous alternate aspects, using either current technology or technology developed after the filing date of this application.

Those of ordinary skill in the art will recognize that a wide variety of modifications, alterations, and combinations can be made with respect to the above described aspects without departing from the scope of the invention, and that such modifications, alterations, and combinations are to be viewed as being within the ambit of the inventive concept.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An artificial intelligence (AI) based system configured to analyze user-specific skin or hair data to predict user-specific skin or hair conditions, the AI based system comprising:
one or more processors;
an scalp and hair analysis application (app) comprising computing instructions configured to execute on the one or more processors; and
an AI based learning model, accessible by the scalp and hair analysis app, and trained with training data regarding scalp and hair regions of respective individuals, the AI based learning model configured to output one or more scalp or hair predictions corresponding to one or more features of the scalp or hair regions of respective individuals,
wherein the training data regarding scalp and hair regions of respective individuals is selected from one or more values corresponding to last wash data of the respective individuals and at least one of: one or more of scalp factors, one or more hair factors, or wash frequency,
wherein the training data comprises data generated with a scalp or hair measurement device configured to determine the one or more features of the scalp or hair regions,
wherein the computing instructions of the scalp and hair analysis app when executed by the one or more processors, cause the one or more processors to:
receive user-specific data of a user, the user-specific data defining a scalp or hair region of the user comprising (1) last wash data of the user, and (2) at least one of: one or more scalp factors of the user, one or more hair factors of the user, or a wash frequency of the user,
analyze, by the AI based learning model, the user-specific data to generate a scalp or hair prediction value corresponding to the scalp or hair region of the user, and
generate, based on the scalp or hair prediction value, a user-specific treatment designed to address at least one feature based on the scalp or hair prediction value of the user's scalp or hair region.

2. The AI based system of claim 1, wherein the one or more scalp factors comprise scalp dryness, scalp oiliness, dandruff, stiffness, redness, unpleasant odor, or itchiness.

3. The AI based system of claim 2, wherein the scalp or hair prediction value comprises a sebum prediction value.

4. The AI based system of claim 1, wherein the scalp or hair region of the user corresponds to one of: (1) a scalp region of the user; or (2) a hair region of the user.

5. The AI based system of claim 1, wherein the one or more hair factors comprise unruliness, hair fall, hair volume, thinning, detangling, hair oiliness, dryness, or hair odor.

6. The AI based system of claim 1, wherein the computing instructions of the scalp and hair analysis app when executed by the one or more processors, further cause the one or more processors to:
generate a quality score based on the scalp or hair prediction value of the user's scalp or hair region.

7. The AI based system of claim 1, wherein the training data comprises image data and non-image data of the respective individuals, and wherein the user-specific data comprises image data and non-image data of the user.

8. The AI based system of claim 7, wherein the image data of the training data and the image data of the user-specific data each comprise one or more sebum images defining an amount of human sebum identifiable within pixel data of the one or more sebum images.

9. The AI based system of claim 1, wherein the computing instructions of the scalp or hair analysis app when executed by the one or more processors, further cause the one or more processors to:
receive an image of the user, the image depicting the scalp or hair region of the user, and
generate a photorealistic representation of the user after virtual application of the user-specific treatment to the scalp or hair region of the user, the photorealistic representation generated by manipulating one or more pixels of the image of the user based on the scalp or hair prediction value.

10. The AI based system of claim 1, wherein the scalp or hair measurement device is configured to determine a sebum level of a skin surface of the user.

11. An artificial intelligence (AI) based method for analyzing user-specific skin or hair data to predict user-specific skin or hair conditions, the AI based method comprising:
receiving, at a scalp and hair analysis application (app) executing on one or more processors, user-specific data of a user, the user-specific data defining a scalp or hair region of the user comprising (1) last wash data of the user, and (2) at least one of: one or more scalp factors of the user, one or more hair factors of the user, or a wash frequency of the user;
analyzing, by an artificial intelligence (AI) based learning model accessible by the scalp and hair analysis app, the user-specific data to generate a scalp or hair prediction value corresponding to the scalp or hair region of the user, wherein the AI based learning model is trained with training data regarding scalp and hair regions of respective individuals and is configured to output one or more scalp or hair predictions corresponding to one or more features of the scalp or hair regions of respective individuals,
wherein the training data regarding scalp and hair regions of respective individuals is selected from one or more values corresponding to last wash data of the respective individuals and at least one of: one or more of scalp factors, one or more hair factors, or wash frequency,
wherein the training data comprises data generated with a scalp or hair measurement device configured to determine the one or more features of the scalp or hair regions; and
generating, by the scalp and hair analysis app based on the scalp or hair prediction value, a user-specific treatment designed to address at least one feature based on the scalp or hair prediction value of the user's scalp or hair region.

12. The AI based method of claim 11, wherein the one or more scalp factors comprise scalp dryness, scalp oiliness, dandruff, stiffness, redness, unpleasant odor, or itchiness.

13. The AI based method of claim 12, wherein the scalp or hair prediction value comprises a sebum prediction value.

14. The AI based method of claim 11, wherein the scalp or hair region of the user corresponds to one of: (1) a scalp region of the user; or (2) a hair region of the user.

15. The AI based method of claim 11, wherein the one or more hair factors comprise unruliness, hair fall, hair volume, thinning, detangling, hair oiliness, dryness, or hair odor.

16. The AI based method of claim 11, the method further comprising:
generating, by the scalp and hair analysis app, a quality score based on the scalp or hair prediction value of the user's scalp or hair region.

17. The AI based method of claim 11, wherein the training data comprises image data and non-image data of the respective individuals, and wherein the user-specific data comprises image data and non-image data of the user.

18. The AI based method of claim 17, wherein the image data of the training data and the image data of the user-specific data each comprise one or more sebum images defining an amount of human sebum identifiable within pixel data of the one or more sebum images.

19. The AI based method of claim 11, the method further comprising:
receiving, at the scalp and hair analysis app, an image of the user, the image depicting the scalp or hair region of the user; and
generating, by the scalp and hair analysis app, a photorealistic representation of the user after virtual application of the user-specific treatment to the scalp or hair region of the user, the photorealistic representation generated by manipulating one or more pixels of the image of the user based on the scalp or hair prediction value.

20. The AI based method of claim 11, wherein the scalp or hair measurement device is configured to determine a sebum level of a skin surface of the user.

21. A tangible, non-transitory computer-readable medium storing instructions for analyzing user-specific skin or hair data to predict user-specific skin or hair conditions, that when executed by one or more processors cause the one or more processors to:
receive, at a scalp and hair analysis application (app) executing on one or more processors, user-specific data of a user, the user-specific data defining a scalp or hair region of the user comprising (1) last wash data of the user, and (2) at least one of: one or more scalp factors of the user, one or more hair factors of the user, or a wash frequency of the user;
analyze, by an artificial intelligence (AI) based learning model accessible by the scalp and hair analysis app, the user-specific data to generate a scalp or hair prediction value corresponding to the scalp or hair region of the user, wherein the AI based learning model is trained with training data regarding scalp and hair regions of respective individuals and is configured to output one or more scalp or hair predictions corresponding to one or more features of the scalp or hair regions of respective individuals,
wherein the training data regarding scalp and hair regions of respective individuals is selected from one or more values corresponding to last wash data of the respective individuals and at least one of: one or more of scalp factors, one or more hair factors, or wash frequency,
wherein the training data comprises data generated with a scalp or hair measurement device configured to determine the one or more features of the scalp or hair regions; and
generate, by the scalp and hair analysis app based on the scalp or hair prediction value, a user-specific treatment designed to address at least one feature based on the scalp or hair prediction value of the user's scalp or hair region.
